(19) United States
(12) Patent Application Publication
(10) Pub. No.: US 2014/0080144 A1
(43) Pub. Date: Mar. 20, 2014

(54) METHOD FOR DETERMINING PLATELET-ASSOCIATED ANALYTES

(71) Applicant: Juergen Patzke, Marburg (DE)

(72) Inventor: Juergen Patzke, Marburg (DE)

(73) Assignee: SIEMENS HEALTHCARE DIAGNOSTICS PRODUCTS GMBH, Marburg (DE)

(21) Appl. No.: 14/030,143

(22) Filed: Sep. 18, 2013

(30) Foreign Application Priority Data
Sep. 18, 2012 (EP) 12184775.0

(51) Int. Cl. G01N 33/86

(52) U.S. Cl. CPC G01N 33/86
USPC 435/7.4; 436/501; 435/13

(57) ABSTRACT

The present invention relates to a routine in vitro method for determining platelet-associated analytes, for example VWF, factor XIII, fibrinogen or D-dimer, in a sample of platelet-rich plasma.

The present invention is in the field of coagulation diagnostics and relates to an in vitro method for determining platelet-associated analytes in a sample.

Physiological processes which, firstly, ensure the fluidity of the blood in the vascular system and, secondly, make sure extravascular blood loss is avoided through the formation of blood clots are covered by the term hemostasis. The regulation of hemostasis involves a multiplicity of protein factors and also cellular components, for example platelets (synonymous: thrombocytes). In the event of vascular injury, there is initially attachment of platelets to the subendothelial collagen. This adhesion is mediated by adhesive proteins, such as von Willebrand factor (VWF). During the adhesion process, the platelets are activated and release mediators from their granules, inducing the aggregation of further platelets and intensification of activation. This achieves primary vascular wall occlusion (primary hemostasis), which is only stabilized by further reactions of the plasmatic coagulation system (secondary hemostasis). Dysregulation of these processes may lead to thrombophilia or bleeding diathesis and, depending on the severity, have life-threatening consequences.

Platelets contain a multiplicity of intracellular ingredients which have physiologically important functions. The most important storage site for various substances, for example platelet factor 4 (PF4), fibrinogen, factor V, factor XIII, von Willebrand factor (VWF), fibronectin, calcium ions and also platelet growth factors, for example PDGF (platelet-derived growth factor), is the alpha granules and the dense granules in the cytoplasm of the platelets. If the platelets are activated, for example as a result of vascular injury, the ingredients are secreted from the granules, initiating in turn important processes for the continuation of the coagulation reaction. Other substances, for example factor XIII, are found in the cytosol of the platelets. Yet other substances are bound in or on the cell membrane of the platelets, in particular receptor proteins such as the ADP receptor, the thromboxane receptor, the thrombin receptor or the glycoproteins Ia, Ib, IIb and IIIa. Moreover, many proteins, for example factor X, factor IX and factor VIII, bind to the cell membrane of the platelets as a result of platelet activation. VWF binds spontaneously to the GPIb receptor of the platelets as a result of shear stress or because of molecular defects. For the VWF-cleaving protease ADAMTS-13, a platelet-associated fraction is likewise known.

Many of these platelet-associated substances and the regulated release thereof have an important role to play in hemostasis. This is also evident from the fact that a range of symptoms is known which can be attributed to disruption of granule function in the platelets. One known condition is the so-called gray platelet syndrome (GPS), a hereditary bleeding disorder caused by reduced or absent alpha granules. Another known condition is the so-called Quebec platelet disorder (QPD), likewise a hereditary bleeding disorder, which is caused by excessive degradation of alpha-granule ingredients. It is therefore of great diagnostic relevance to identify deficiencies or dysfunctionalities of platelet-associated substances and to identify defects in the release of said substances from the platelet granules.

A further known condition is the so-called von Willebrand disease (VWD), a hereditary or acquired bleeding disorder. Different types of von Willebrand disease are distinguished, since it can be caused by various VWF disorders. In type 3 VWD, the VWF antigen (VWF:Ag) is completely absent from both the plasma and the alpha granules of the platelets. In type 1 VWD, which is notable for a reduced plasmatic VWF antigen concentration, two subtypes are distinguished which differ in that the alpha-granule VWF antigen concentration in the platelets is likewise reduced in one subtype, whereas the alpha-granule VWF antigen concentration in the platelets is normal in the other subtype.

Since various therapies are used for the different types of von Willebrand disease, each individual case requires a very accurate classification. It is therefore desirable to determine not only the VWF antigen concentration or activity in the plasma of a patient, but also the VWF antigen concentration or activity in the platelets of the patient.

Known methods for determining platelet-associated substances are technically complex and not suitable for routine processing in a clinical laboratory. The fundamental problem is that many diagnostically relevant platelet-associated substances, for example VWF, occur not only in the platelets, but also in the plasma. To analyze intracellular platelet analytes, platelet lysates are usually prepared from fresh washed platelets. To this end, platelet-rich plasma is repeatedly suspended in a wash solution to remove the plasma constituents, and centrifuged, and the platelets are then lysed by adding a detergent-containing buffer. In the plasma-free platelet lysate thus obtained, the amount or the activity of a desired analyte is then determined.

De Romeuf & Mazurier describe a simplified method for determining platelet VWF, in which at least the wash steps are dispensed with. In the simplified method, platelet-rich plasma is centrifuged only once, the supernatant is carefully removed, and the cell pellet is resuspended in a detergent-containing buffer. In the platelet lysate thus obtained, the amount or the activity of the platelet VWF is then determined (De Romeuf, C. & Mazurier, C. Interest of a simple and fast method for platelet von Willebrand factor characterization. Thrombosis Research 1996, 83(4): 287-298).

This simplified method is not suitable either for routine processing in a clinical laboratory and, in particular, not for an automated assay procedure, since the sample preparation still requires that the sample material (platelet-rich plasma) be centrifuged and the supernatant be carefully removed. No systems are known, at least in the field of automated coagulation analyzers, which could centrifuge a sample and remove the supernatant precisely from a cell pellet.

It is therefore an object of the present invention to provide a method for determining the amount or the activity of a platelet-associated analyte in a sample, which method dispenses with complex sample preparation, in particular additional centrifugation and wash steps, and which method is suitable for routine processing in a clinical laboratory and, in particular, for processing on an automated assay system.

The object is solved by carrying out a method comprising the following steps:
a) providing a first assay volume containing platelet-rich plasma from an individual and a detergent,
b) providing a second assay volume containing
   i) platelet-rich plasma from the individual and no detergent or
   ii) platelet-poor plasma from the individual and no detergent or
   iii) platelet-poor plasma from the individual and a detergent,
c) measuring the amount or the activity of the analyte in the first and in the second assay volume and
d) comparing the measurement results,
   wherein the difference between the measurement results corresponds to the amount or the activity of the platelet-associated analyte.

The term “platelet-associated analyte” is to be understood in a broad sense and encompasses intracellular ingredients of platelets, for example ingredients of the platelet granules or of the cytosol, extracellular substances which adhere to the outer platelet surface, for example VWF or fibrinogen, and membrane constituents which are anchored in the cell membrane of the platelets, for example transmembrane receptors. The term “platelet-associated analyte” encompasses all types of substances, for example peptides, proteins (e.g., factor XIII, VWF, GPIb receptor, fibrinogen, D-dimer), lipids, ions (e.g., calcium ions), nucleotides (e.g., ADP and ATP), nucleic acids (e.g., DNA, RNA and miRNA) and long-chain polyphosphates (n>50), which are present in isolated, preferably human platelets.

The term “platelet-rich plasma” (PRP) encompasses a plasma sample from an individual, preferably a plasma sample from a human individual, which contains at least 10.000 platelets per microliter [μL] of sample. Various methods for obtaining platelet-rich plasma from a whole blood sample are known to a person skilled in the art. A customary method works as follows: an anticoagulated whole blood sample is centrifuged at 170 g for 15 minutes or at 180 g for 10 minutes. This achieves the formation of a lower layer composed of red and white blood cells and an upper layer of platelet-rich plasma. The latter is carefully removed without being mixed with the lower layer.

Platelet-poor plasma (PPP) by contrast, which is usually meant when the term “plasma” is used, is ideally platelet-free, but must in any case contain fewer than 10.000 platelets per microliter [μL] of sample. Various methods for obtaining platelet-poor plasma from a whole blood sample are known to a person skilled in the art. A customary method works as follows: an anticoagulated whole blood sample is centrifuged at 1500 g for at least 15 minutes. This achieves the formation of a lower layer composed of red and white blood cells and of platelets and an upper layer of platelet-poor plasma. The latter is carefully removed without being mixed with the lower layer.

The term “detergent” encompasses surface-active substances, in particular nonionic surfactants, which are capable of increasing the solubility of cellular lipid membranes in an aqueous medium. It is known that detergents permeabilize cellular membranes, and so intracellular constituents or transmembrane constituents are released into the surrounding medium or are more easily accessible for binding partners which are used for detecting the cellular constituents. Examples of suitable detergents for permeabilizing cellular lipid membranes or for lysing cells, including platelets for example, are polyethylene glycol p-(1,1,3,3-tetramethylbutyl)-phenyl ether (trade name: TRITON® X-100), polyoxyethylene (20) sorbitan monolaurate (trade name: TWEEN® 20), polidocanol (trade name: THESIT®; synonyms: polyethylene glycol dodecyl ether, polyethylene glycol 400 dodecyl ether), glycerol, saponin, digitonin, filipin, octyl glucoside, dodecyl sulfates, Zonyl® and deoxycholate.

The method according to the invention for determining the amount or the activity of a platelet-associated analyte in a sample from an individual comprises providing a first assay volume containing platelet-rich plasma from the individual and a detergent. Preferably, the detergent is added to the platelet-rich plasma in such an amount that the detergent is present in the assay volume in a proportion by volume of from 0.1 to 1.6%, particularly preferably from 0.2 to 0.8%. By adding analyte-specific detection reagents, for example labeled binding partners specific for the analyte to be detected, it is possible to determine the amount or the activity of the analyte in the assay volume. The amount or activity of the analyte determined in the first assay volume corresponds to the sum of the amount or activity of the analyte which is present in the plasma (plasma fraction), and the amount or activity of the same analyte which is present in or on the platelets (platelet fraction).

The method according to the invention further comprises providing a second assay volume comprising
i) platelet-rich plasma from the individual and no detergent or
ii) platelet-poor plasma from the individual and no detergent or
iii) platelet-poor plasma from the individual and a detergent.

Preferably, in alternative iii), the detergent is added to the platelet-poor plasma in such an amount that the detergent is present in the assay volume in a final concentration at a proportion by volume of from 0.1 to 1.6%, particularly preferably in a proportion by volume of from 0.2 to 0.8%. Particularly preferably, in alternative iii), the detergent is added to the platelet-poor plasma in an amount such that the detergent is present in the same final concentration as in the first assay volume. By adding the same analyte-specific detection reagents as in the first assay volume, for example labeled binding partners specific for the analyte to be detected, it is possible to determine the amount or the activity of the analyte in the second assay volume. The amount or activity of the analyte determined in the second assay volume corresponds to the amount or activity of the analyte which is present in the plasma (plasmatic analyte). The amount or activity of the same analyte which is present in the platelets (platelet-associated analyte) is not determined because either no detergent is used which would make the platelet fraction of the analyte accessible (alternative i) or only platelet-poor plasma is used which contains an insufficient amount of platelets (alternatives ii and iii).

The method according to the invention further comprises comparing the measurement results, i.e., comparing the measurement result for the first assay volume (amount or activity of the plasmatic analyte and of the platelet-associated analyte) with the measurement result for the second assay volume (amount or activity of the plasmatic analyte). The difference between the measurement results corresponds to the amount or the activity of the platelet-associated analyte. For example, by subtracting the measurement result for the second assay volume from the measurement result for the first assay volume, it is possible to determine the amount or activity of the platelet-associated analyte.

The amount or activity of the analyte in the first and in the second assay volume can be measured using any detection method which is suitable for detecting the analyte.

The amount or activity of the analyte can be measured in, for example, a heterogeneous binding assay. Heterogeneous binding assays are notable for one or more separation steps and/or wash steps. Separation can, for example, be achieved by immunoprecipitation, precipitation with substances such as polyethylene glycol or ammonium sulfate, filtration, magnetic separation, or binding to a solid phase. Such a solid phase consists of porous and/or nonporous, generally water-insoluble material. It can exhibit a very wide variety of different forms, for example: vessel, tube, microtiter plate, bead, microparticle, rod, strip, filter or chromatography paper, etc. In the case of heterogeneous binding assays in sandwich format, generally a first analyte-specific binding partner, for example a monoclonal or polyclonal antibody or an analyte-binding antibody fragment or an oligonucleotide, is bound to a solid phase and is used to remove the binding complex of analyte/analyte-specific binding partner from the liquid phase, whereas a second analyte-specific binding partner for detecting the binding complex bears a detectable label (e.g., an enzyme, a fluorescence label or chemiluminescence label, etc.). These assay methods are further divided into so-called one-step sandwich assays, in which the two specific binding partners are incubated simultaneously with the sample, and two-step sandwich assays, in which the sample is first incubated with the solid-phase reagent and is incubated with the detection reagent after a separation and wash step involving the solid-phase-bound binding complex composed of analyte and analyte-specific binding partner. A classic example of a heterogeneous binding assay is the ELISA assay.

The amount or activity of the analyte can also be measured in a homogeneous binding assay. In homogeneous binding assays, there is no separation of free and bound analyte-specific binding partners. The assay volume containing the analyte-specific binding partners, the signal-generating components and the sample is measured after, or even during, the binding reaction without a further separation or wash step and the corresponding measurement signal is determined. Examples of homogeneous immunoassays are many turbidimetric or nephelometric methods, in which the analyte-specific binding partners used for detection can be associated with latex particles. Examples of homogeneous assay formats are EMIT® assays, CEDIA® assays, fluorescence polarization immunoassays, luminescent oxygen channeling immunoassays (“LOCI”; see EP-A2-515194), T2MR (T2 Biosystems, Inc.; see US-A1-20120164644), etc. In a homogeneous sandwich immunoassay, for example a nephelometric latex assay, the binding partners, for example monoclonal or polyclonal antibodies or an analyte-binding antibody fragment or an oligonucleotide, are incubated together with the sample and the signal is measured during and/or after the incubation, without a separation or wash step being carried out before the measurement.

In a preferred embodiment of the method according to the invention, the amount or activity of the analyte in the first and the second assay volume is measured using a latex agglutination test. To this end, each assay volume is mixed with a particulate solid phase, preferably with latex particles, which is associated with at least one analyte-specific binding partner, and the agglutination of the particulate solid phase is optically measured. Alternatively, each assay volume is mixed with magnetic particles having, on the surface, binding partners which alter the aggregation of the particles in the presence of the analyte. The reaction volume is exposed to a magnetic field, and the agglutination of the particulate solid phase is determined on the basis of the T2 relaxation rate of the reaction volume.

The amount or activity of the analyte can also be measured in a competitive binding assay. In a competitive binding assay, sample analyte and reagent analyte, for example a labeled analyte, compete for binding to a limited number of analyte-specific binding partners. Examples to illustrate the principle: (i) sample analyte competes with reagent analyte associated with a component of a signal-generating system, for binding to solid-phase-associated analyte-specific binding partners or (ii) sample analyte competes with solid-phase-associated reagent analyte for binding to analyte-specific binding partners associated with a component of a signal-generating system.

In a preferred embodiment of the method according to the invention, the amount or the activity of platelet-associated VWF is measured. The amount of VWF antigen (VWF:Ag) in an assay volume can be measured using a heterogeneous or homogeneous binding assay in which at least one VWF-specific binding partner, preferably a monoclonal or polyclonal antibody or a VWF-binding antibody fragment, is used. VWF activity (VWF Ac) in an assay volume can be measured using a classic ristocetin cofactor assay (VWF:Rco). To this end, the assay volume is mixed with ristocetin and fixed platelets and the agglutination of the platelets is determined. Alternatively, VWF activity can also be determined on the basis of the capacity of VWF to bind to GPIb. To this end, the assay volume is mixed either with ristocetin and wild-type, typically recombinant GPIb protein or a GPIb protein fragment which is bound to a solid phase (WO-A2-01/02853), or without ristocetin but with a mutated, typically recombinant GPIb protein or a GPIb protein fragment which is bound to a solid phase (WO-A2-2009/007051, WO-A1-2009/026551), and the binding of VWF to the GPIb protein is quantified. Alternatively, VWF activity can also be determined on the basis of the capacity of VWF to bind to collagen.

In another preferred embodiment of the method according to the invention, the amount or the activity of platelet-associated factor XIII is measured. The amount of factor XIII in an assay volume can be measured using a heterogeneous or homogeneous binding assay in which at least one factor XIII-specific binding partner, preferably a monoclonal or polyclonal antibody or a factor XIII-binding antibody fragment, is used. Factor XIII activity in an assay volume can be measured using an assay in which factor XIII in the assay volume is activated with thrombin in the presence of Ca2+ ions to form factor XIIIa. The assay volume is then mixed with a synthetic, glutamine-containing peptide and with glycine ethyl ester, which serve as substrates for the formation of intermolecular amide bonds owing to factor XIIIa. In order to quantitatively detect the ammonia released in this reaction, the sample is additionally mixed with NADH (nicotinamide adenine dinucleotide hydride), NADPH or thio-NAD(P)H and with components of an NADH-dependent indicator reaction, viz. with glutamate dehydrogenase (GLDH) and α-ketoglutarate. In the presence of ammonia, GLDH converts α-ketoglutarate into glutamate. This reaction additionally consumes NADH, forming NAD+, the oxidized form of NADH. NAD+ has an absorption spectrum different to that of NADH, and so the absorption of the assay volume changes proportionally to NADH consumption and thus proportionally to ammonia amount and thus proportionally to factor XIII amount or activity (EP-A2-336353; WO-A1-2011/042071).

In another preferred embodiment of the method according to the invention, the amount of platelet-associated D-dimer is measured. The amount of D-dimer in an assay volume can be measured using a heterogeneous or homogeneous binding assay in which at least one D-dimer-specific binding partner, preferably a monoclonal or polyclonal antibody or a D-dimer-binding antibody fragment, is used.

### FIGURE DESCRIPTION

FIG. 1 is a graph showing factor XIII activity (as % of the norm) in samples of platelet-poor and platelet-rich plasma from 3 healthy donors (donors 1, 2 and 3) as per exemplary embodiment 2. Bars 1-4 each show the assay results for a sample of platelet-poor plasma (PPP sample). Bars 5-8 each show the assay results for a sample of platelet-rich plasma (PRP sample). Bars 1 and 5 represent assay results determined in the complete absence of detergent. The remaining bars represent assay results determined in the presence of detergent (Thesit) in the assay volume (2 and 6: pretreatment of the sample with Thesit/no Thesit in assay reagents; 3 and 7: no pretreatment of the sample with Thesit/Thesit in assay reagents; 4 and 8: pretreatment of the sample with Thesit, and Thesit in assay reagents). A comparison of bar 5 (PRP as sample, and determination of the assay results in the absence of detergent) with bars 6-8 (PRP as sample, and determination of the assay results in the presence of detergent) shows that a significantly higher factor XIII activity is measured which can be attributed to the activity of platelet-associated factor XIII.

### EXAMPLES

### Example 1

### Determination of the Activity of Platelet-Associated VWF

Preparation of Platelet-Rich Plasma (PRP):

10 mL of fresh citrated whole blood from each of four apparently healthy donors were centrifuged in the original collection tubes at 180 g for 10 minutes. The platelet-rich plasma in the supernatant was carefully removed using a pipet and kept sealed at room temperature.

Preparation of Platelet-Poor Plasma (PPP):

From the preparation of the platelet-rich plasma, the lower layer which remained in the collection tubes and which contains the red and white blood cells, remnants of thrombocytes and plasma was centrifuged at 2000 g for 20 minutes. The clear supernatant, i.e., the platelet-poor plasma, was carefully removed using a pipet and kept sealed at room temperature.

### Example 1.1

### Ristocetin Cofactor Assay for Determining the VWF Activity of Platelet-Associated VWF

The above-described platelet-rich and platelet-poor plasma from the four donors was used as sample. To prepare a first assay volume, 10 μL of sample were mixed in each case with 20 μL of NaCl solution (0.9%) and 150 μL of a reagent containing about 1.2 million fixed thrombocytes per μL, ristocetin (1.25 mg/mL) and 0.75 percent by volume Thesit (Sigma-Aldrich, Munich, Germany), yielding in the assay volume a proportion by volume of Thesit of 0.63%. To prepare a second assay volume without detergent, 10 μL of sample were mixed with 20 μL of NaCl solution (0.9%) and 150 μL of a reagent containing about 1.2 million fixed thrombocytes per μL and ristocetin (1.25 mg/mL). Platelet aggregation in each assay volume was determined on the basis of an absorbance measurement. The aggregation reaction is proportional to the VWF activity of the sample.

The determined VWF activities (VWF:RCo as % of the norm) of the 4 platelet-rich and the 4 platelet-poor plasma samples were averaged in each case. The averaged VWF activities ([%], % of the norm) are shown in table 1.

TABLE 1
Averaged VWF-RCo activity (% of the norm) of platelet-
poor and platelet-rich plasma in the absence and
in the presence of a detergent (Thesit)

| | VWF:RCo (%) in the | VWF:RCo (%) in the |
|---|---|---|
| | absence of Thesit | presence of Thesit |
| Platelet-poor plasma | 89.8 | 95.6 |
| (PPP) | | |
| Platelet-rich plasma | 95.5 | 121.8 |
| (PRP) | | |

To determine the activity of platelet-associated VWF, one of the following can then be carried out:
a) calculating the difference between the VWF activity determined in platelet-rich plasma in the presence of detergent and the VWF activity determined in platelet-rich plasma in the absence of detergent, i.e., in this specific case:

121.8%−95.5%=26.3% or
b) calculating the difference between the VWF activity determined in platelet-rich plasma in the presence of detergent and the VWF activity determined in platelet-poor plasma in the absence of detergent, i.e., in this specific case:

121.8%−89.8%=32.0% or
c) calculating the difference between the VWF activity determined in platelet-rich plasma in the presence of detergent and the VWF activity determined in platelet-poor plasma in the presence of detergent, i.e., in this specific case:

121.8%−95.6%=26.2%.

The thus determined VWF activity for platelet-associated VWF in the range of from 26.2 to 32.0% (absolute) is plausible for healthy donors, since values of 10-25% of total VWF are reported in the literature for platelet VWF (McGrath, R. T. et al. Platelet von Willebrand factor structure, function and biological importance. British Journal of Haematology 2010, 148, 834-843). The VWF activity which is increased by 5.8% and which is measured in platelet-poor plasma in the presence of Thesit relative to the measurement in the absence of Thesit is minimal and can be attributed to either the imprecision of the assay or a negligible influence of the detergent on the assay system.

### Example 1.2

### Latex Agglutination Test for Determining the VWF Activity of Platelet-Associated VWF

The INNOVANCE® VWF Ac assay (Siemens Healthcare Diagnostics Products GmbH, Marburg, Germany) is a latex agglutination test for determining VWF activity in plasma samples. The above-described platelet-rich and platelet-poor plasma from the four donors was used as sample. To determine VWF activity, 15 μL of sample, 30 μL of Owren's Veronal buffer, 70 μL of reaction buffer (which contained, inter alia, 0.90% proportion by volume of the detergent Thesit), 13 μL of GPIbα reagent (buffer solution containing isolated GPIbα protein which binds to VWF in the absence of ristocetin) and 40 μL of latex reagent (buffer solution containing latex particles coated with a monoclonal anti-GPIbα antibody) were mixed in each case, and particle agglutination was determined turbidimetrically. In the assay volumes, there was therefore a proportion by volume of 0.38% Thesit. The determined VWF activities (as % of the norm) of the 4 platelet-rich and the 4 platelet-poor plasma samples were averaged in each case. The averaged VWF activities ([%], % of the norm) are shown in table 2.

TABLE 2
Averaged VWF activity (% of the norm) of platelet-poor and platelet-
rich plasma in the presence of a detergent (Thesit)

| | VWF (%) |
|---|---|
| | in the presence of Thesit |
| Platelet-poor plasma (PPP) | 102.9 |
| Platelet-rich plasma (PRP) | 121.8 |
| Platelet-associated VWF | 18.9 |

The difference between the VWF activity determined in platelet-rich plasma in the presence of detergent and the VWF activity determined in platelet-poor plasma in the presence of detergent, i.e., in this specific case:

121.8%−102.9%=18.9%,

corresponds to platelet-associated VWF.

### Example 2

### Determination of the Activity of Platelet-Associated Factor XIII

F XIII activity was determined using the Berichrom® FXIII assay (Siemens Healthcare Diagnostics Products GmbH, Marburg, Germany). F XIII present in the sample is activated by adding thrombin to the sample to form F XIIIa. Fibrin formed by thrombin likewise speeds up this reaction. Fibrinogen is not removed before measurement, since this is associated with a loss of F XIII. Instead, fibrin formed by thrombin action is prevented by an aggregation-inhibiting peptide from forming a clot and is kept in solution. F XIIIa connects a specific peptide substrate with glycine ethyl ester, releasing ammonia. This is determined in an enzymatic reaction running in parallel. The decrease in NADH is measured via the absorbance at 340 nm. When results were above the initial measurement range, a 1:2 predilution of the sample was carried out in a phosphate buffer and measurement was carried out again.

The above-described platelet-rich and platelet-poor plasma from three healthy donors was used as sample. 15 μL of sample were in each case mixed with 75 μL of Berichrom FXIII activator reagent (containing, inter alia, thrombin, and with Thesit (1.49 percent by volume) or without Thesit) and incubated. Thereafter, 75 μL of the Berichrom FXIII detection reagent (containing, inter alia, GLDH (20 IU/mL), a synthetic F XIII peptide substrate (2.4 g/L), ADP, glycine ethyl ester (1.4 g/L) and α-ketoglutarate (2.7 g/L)) were added, and the color reaction was started. This yielded in the assay volume a proportion by volume of 0.68% Thesit.

In an additional experiment, the samples were pretreated with detergent. To this end, the samples were incubated in the presence of a proportion by volume of 0.63% Thesit for 30 minutes at 37° C. before they were mixed with the reagents to determine factor XIII activity.

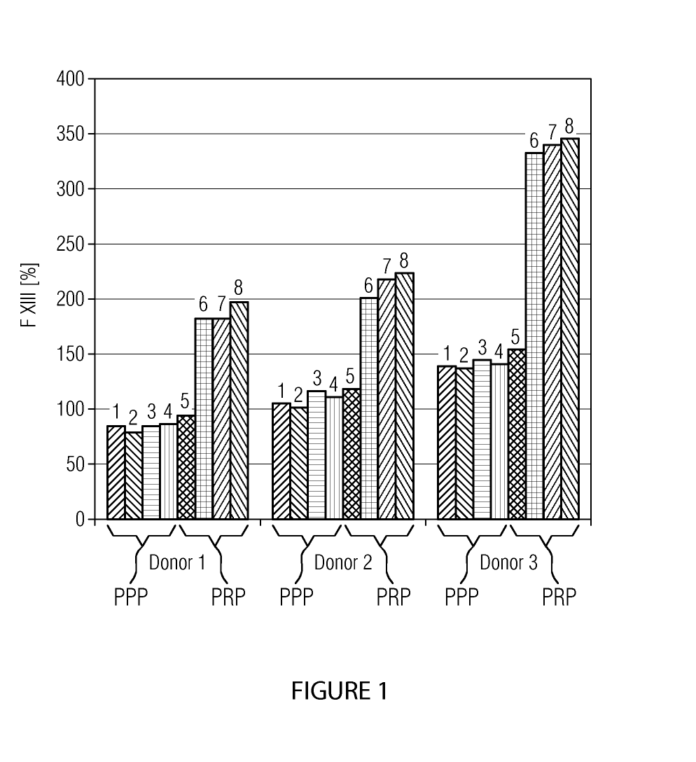

The determined factor XIII activities (as % of the norm) of the platelet-rich and the platelet-poor plasma samples from the 3 donors are shown in FIG. 1.

The factor XIII activity of the PPP sample from a donor—irrespective of with which assay volume the activity was determined—and the factor XIII activity of the PRP sample from a donor in the absence of detergent are in each case very similar. This provides evidence that the presence of detergent, pretreatment with detergent, and the presence of thrombocytes in the assay volume do not interfere with the factor XIII assay. In both the pretreatment of a PRP sample with detergent and the use of detergent in an assay reagent—in short: whenever the assay method is carried out in the presence of detergent—a large amount of platelet-associated factor XIII is released in platelet-rich plasma. This shows that the method according to the invention captures the total amount of releasable platelet-associated factor XIII in addition to plasma factor XIII.

The platelet-associated fraction of factor XIII can be determined by calculating the difference between the total amount and the plasma fraction. This yields, for the 3 measured samples, the values in table 3 for the platelet-associated factor XIII fraction.

TABLE 3
Factor XIII activities (% of the norm) of platelet-
associated factor XIII calculated from the difference
between the measurement values of a first assay volume
and various alternative second assay volumes

| | Donor 1 | Donor 2 | Donor 3 |
|---|---|---|---|
| | Total factor XIII (%) | | |
| PRP as sample, assay with | 181.6 | 217.4 | 312.6 |
| detergent | | | |
| | Platelet-associated factor XIII (%) | | |
| Plasma (PPP) as sample, assay | 97.8 | 112.7 | 173.7 |
| without detergent | | | |
| Plasma (PPP) as sample, assay | 98.2 | 101.2 | 169.3 |
| with detergent | | | |
| PRP as sample, assay without | 87.2 | 99.7 | 161.0 |
| detergent | | | |

The content of platelet-associated factor XIII is considerably higher for donor No. 3 than for donors Nos. 1 and 2.

1. A method for determining the amount or the activity of a platelet-associated analyte in a sample from an individual, wherein the method comprises the following steps:
a) providing a first assay volume containing platelet-rich plasma from the individual and a detergent,
b) providing a second assay volume containing
i) platelet-rich plasma from the individual and no detergent or
ii) platelet-poor plasma from the individual and no detergent or
iii) platelet-poor plasma from the individual and a detergent,
c) measuring the amount or the activity of the analyte in the first and in the second assay volume and
d) comparing the measurement results,
wherein the difference between the measurement results corresponds to the amount or the activity of the platelet-associated analyte.

2. The method as claimed in claim 1, wherein the detergent is a nonionic surfactant, preferably from the group consisting of polyethylene glycol p-(1,1,3,3-tetramethylbutyl)-phenyl ether (TRITON X-100), polyoxyethylene (20) sorbitan monolaurate (TWEEN 20) and polidocanol (THESIT).

3. The method as claimed in claim 1, wherein the detergent is present in the assay volume in a final concentration at a proportion by volume of from 0.1 to 1.6%, preferably in a final concentration in a proportion by volume of from 0.2 to 0.8%.

4. The method as claimed in claim 1, wherein the amount or the activity of the analyte in the first and in the second assay volume is measured by mixing each assay volume with a particulate solid phase, preferably with latex particles, which is associated with at least one analyte-specific binding partner, and wherein the agglutination of the particulate solid phase is measured.

5. The method as claimed in claim 1, for determining the amount or the activity of a platelet-associated analyte from the group consisting of von Willebrand factor, factor XIII, D-dimer, fibrinogen and polyphosphates.
